(19) United States
(12) Patent Application Publication
(10) Pub. No.: US 2008/0081326 A1
(43) Pub. Date: Apr. 3, 2008

(54) Methods and devices for diagnostic testing

(76) Inventor: Jun Amano, Hillsborough, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/542,504

(22) Filed: Oct. 3, 2006

(51) Int. Cl. C12Q 1/70; G01N 33/574; G01N 33/569; G01N 33/554; G01N 33/543; G01N 33/53

(52) U.S. Cl. 435/5; 435/7.31; 435/7.32; 435/7.23; 436/518; 977/902; 435/287.2

(57) ABSTRACT

Methods, devices and apparatus are disclosed for analyzing a sample for the presence of one or more analytes. A sample is contacted with a well comprising a plurality of p-n junction nanowire pairs. One member of the nanowire pair comprises a capture moiety, and one member of the nanowire pair is an excitation nanowire and the other member is a detection nanowire. The contacting is carried out under conditions for binding of an analyte to a respective binding partner. The excitation nanowire is employed to excite a luminescent label bound to the capture moiety and the detection nanowire is used to detect a signal resulting from excitation of the luminescent label. The amount of the signal is related to the presence and/or amount of an analyte in the sample.

### BACKGROUND

The present invention relates to methods and apparatus for carrying out highly sensitive analyses for materials of interest and, more particularly, for carrying out such analyses in channels of a microfluidic system.

The clinical diagnostic field has seen a broad expansion in recent years, both as to the variety of materials of interest that may be readily and accurately determined, as well as the methods for the determination. Convenient, reliable and non-hazardous means for detecting the presence of low concentrations of materials in liquids is desired. Some materials of interest may be present in body fluids in concentrations below 10⁻¹² molar. The difficulty of detecting the presence of these materials in low concentrations is enhanced by the relatively small sample sizes that can be utilized.

The need to determine multiple analytes in biological fluids has become increasingly apparent in many branches of medicine. In endocrinology the knowledge of plasma concentration of a number of different hormones is often required to resolve a diagnostic problem or a panel of markers for a given diagnosis where the ratios could assist in determining disease progression. Other areas of interest include, for example, cancer antigen screening, allergy testing, screening of transfused blood for viral contamination or genetic diagnosis and so forth.

Any one of a number of infectious agents may cause some pathological disease states. In other cases the diagnosis and assessment of disease states may be best evaluated by the measurement of a number of analytes in a sample, such as a panel of cytokines and chemokines, a panel of tissue specific disease markers, a panel of diagnostic antibodies and antigens and the like. Another example for the utility of simultaneous analysis of multi-analytes is the determination of the level of expression of a panel of genes in a given cell population or the simultaneous detection and quantification of multiple nucleic acid sequences in a single sample. Other benefits of simultaneous detection and quantification of multiple analytes include the ability to incorporate internal controls to the test sample.

Microfluidic systems have been developed for performing chemical, clinical, and environmental analysis of chemical and biological specimens. The term microfluidic system refers to a system or device having a network of chambers connected by channels, in which the channels have microscale features, that is, features too small to examine with the unaided eye, e.g., having at least one cross-sectional dimension in the range from about 0.1 μm to about 1 mm. Such microfluidic systems are often fabricated using photolithography, wet chemical etching, and other techniques similar to those employed in the semiconductor industry. The resulting devices can be used to perform a variety of sophisticated chemical and biological analytical techniques.

It is desirable to provide structures, systems, and methods that provide highly sensitive, low cost analyses for point of care applications as well as for diagnostic instrumentation.

### SUMMARY

An embodiment of the present invention is a method for analyzing a sample for the presence of one or more analytes. A sample is contacted with a well comprising a plurality of p-n junction nanowire pairs. One member of the nanowire pair comprises a capture moiety and one member of the nanowire pair is an excitation nanowire and the other member is a detection nanowire. The member of the nanowire pair comprising the capture moiety may be the excitation nanowire or the detection nanowire, which will determine the property of the other member of the nanowire pair. The contacting is carried out under conditions for binding of an analyte to a respective binding partner. The excitation nanowire is employed to excite a luminescent label bound to the capture moiety and the detection nanowire is used to detect a signal resulting from excitation of the luminescent label. The signal is related to the presence and/or amount of an analyte in the sample.

In some embodiments of the above method, the member of the nanowire pair comprising the capture moiety is the excitation nanowire and the other member of the nanowire pair is the detection nanowire. The method comprises increasing the bias voltage of the excitation nanowire sufficient to excite the luminescent label and detecting luminescence emitted by the luminescent label by means of detecting photocurrent using the detection wire.

In some embodiments of the above method, the member of the nanowire pair comprising the capture moiety is the detection nanowire and the other member of the nanowire pair is the excitation nanowire. The method comprises increasing the bias voltage of the excitation nanowire to excite the luminescent label and detecting photocurrent by means of the detection nanowire.

Another embodiment of the present invention is a method for analyzing a sample for the presence of one or more analytes. The sample is contacted with a well comprising a plurality of p-n junction nanowire pairs. One member of the nanowire pair comprises a capture moiety. The contacting is carried out under conditions for binding of an analyte to a respective capture moiety. A light emitting property of the one of the members of the nanowire pair and a detection property of the other member of the nanowire pair are employed to determine the presence and/or amount of an analyte in the sample.

In some embodiments the capture moiety is a binding partner for one of the respective analytes and each of the analytes bound to a respective capture moiety comprises a luminescent label. The method comprises increasing the bias voltage of the p-n junction nanowire comprising the capture moiety such that light is emitted sufficient to excite each of the luminescent labels and detecting the amount of luminescence emitted from the luminescent labels by means of detecting photocurrent using the other member of the p-n junction nanowire pair. The luminescence is related to the presence and/or amount of one or more of the analytes in the sample.

In some embodiments the capture moiety is a binding partner for one of the respective analytes and each of the analytes bound to a respective capture moiety comprises a luminescent label. The method comprises increasing the bias voltage of the other member of the p-n junction nanowire pairs such that light is emitted sufficient to excite each of the luminescent labels on the member of the p-n junction nanowire pairs comprising the capture moiety and detecting photocurrent by means each member of the p-n junction nanowire pair comprising a capture moiety. The photocurrent is related to the presence and/or amount of one or more of the analytes in the sample.

Another embodiment of the present invention is a method for analyzing a sample for the presence of one or more analytes. The method comprises contacting the sample with a channel of a microfluidic system wherein the channel comprises a plurality of p-n junction nanowire pairs. One member of the nanowire pair comprises a capture moiety that is a binding partner for an analyte. The contacting is carried out under conditions for binding of an analyte to a respective binding partner. Each of the analytes comprises a luminescent label. The bias voltage of one of the members of the p-n junction nanowire pairs is increased such that light is emitted sufficient to excite each of the luminescent labels. The amount of luminescence emitted from each of the luminescent labels is determined by means of a photocurrent detection property of one of the members of the nanowire pairs. The amount of luminescence is related to the presence and/or amount of one or more of the analytes in the sample.

Another embodiment of the present invention is a device for analyzing a sample for the presence of one or more analytes. The device comprises (a) a well comprising a plurality of p-n junction nanowire pairs wherein one member of each of the nanowire pairs comprises a capture moiety for one of the respective analytes and wherein one member of each of the nanowire pairs is a photocurrent detector and the other member is a light emitter, and (b) a mechanism for varying the bias voltage of one member of the p-n junction nanowire pairs such that excitation light is emitted.

Another embodiment of the invention is an apparatus comprising a device as described above, a computer system for controlling the mechanism for varying the bias voltage of the p-n junction nanowires, and a computer program on a computer readable medium for controlling the computer.

### BRIEF DESCRIPTION OF THE DRAWINGS

The following figures are included to better illustrate the embodiments of the apparatus and technique of the present invention. The figures are not to scale and some features may be exaggerated for the purpose of illustrating certain aspects or embodiments of the present invention.

FIG. 1 is a perspective view of a microfluidic system including a microfluidic device in accordance with one embodiment of the invention.

FIG. 2 is a perspective view of a portion of a microfluidic channel of the microfluidic system of FIG. 1.

### DETAILED DESCRIPTION OF SPECIFIC EMBODIMENTS

Before describing the present invention in detail, it is to be understood that this invention is not limited to particular devices or biological systems, which can, of course, vary. It is also to be understood that the terminology used herein is for the purpose of describing particular embodiments only and is not intended to be limiting. As used in this specification and the appended claims, the singular forms “a”, “an” and “the” include plural referents unless the content clearly dictates otherwise. As used herein, the phrase “at least” means that the indicated item is equal to or greater than that designated value and the term “about” means that the designated value may vary by plus or minus ten percent, or nine percent, or eight percent, or seven percent, or six percent, or five percent, or four percent, or three percent, or two percent, or one percent. The term “substantially” varies with the context as understood by those skilled in the relevant art and generally means at least 70%, preferably means at least 80%, more preferably at least 90%, and most preferably at least 95%.

Embodiments of the present invention provide methods and devices for conducting point of care diagnostic testing as well as instrumental analyses. The methods and devices of some embodiments of the present invention comprise semiconductor p-n junction nanowires that function as electrochemical detectors and/or as an optical light sources for detection of luminescence. With embodiments of the present invention, it is possible to carry out electrochemical and/or luminescent detection of analytes in a relatively simple device. The devices have a relatively small size and are lightweight and easily transportable, thus rendering the devices suitable for point-of-care applications. Because of relative small size, embodiments of the devices are particularly suited for conducting analyses on relatively small amounts of sample. Embodiments of the methods and devices have a sensitivity that is comparable to laboratory diagnostics. While particularly suited for point of care applications, some embodiments of the present invention are also applicable to diagnostic instrumentation.

As discussed above, some embodiments of the present invention are directed to methods for analyzing a sample for the presence of one or more analytes. A sample is contacted with a well comprising a plurality of p-n junction nanowire pairs. One member of the nanowire pair comprises a capture moiety, and one member of the nanowire pair is an excitation nanowire and the other member is a detection nanowire. The contacting is carried out under conditions for binding of an analyte to a respective binding partner. The excitation nanowire is employed to excite a luminescent label bound to the capture moiety and the detection nanowire is used to detect a signal resulting from excitation of the luminescent label. The signal, and in many embodiments the amount of the signal, is related to the presence and/or amount of an analyte in the sample.

### Devices

Embodiments of devices in accordance with the present invention comprise a well having contained therein a plurality of p-n junction nanowire pairs. In some embodiments the nanowires are attached to surfaces or walls of the well and lie substantially horizontally across the well when the walls are substantially vertical and opposite to one another.

A nanowire is a wire of dimensions of the order of nanometers (10⁻⁹ meters). Accordingly, a nanowire is a structure having at least one region or characteristic dimension with a dimension of less than about 500 nm, less than about 200 nm, less than about 100 nm, less than about 50 nm, or even less than about 20 nm, and the like, and has an aspect ratio (for example, length:width) of greater than about 10, preferably, greater than about 50, and more preferably, greater than about 100, and the like. In many cases, the region or characteristic dimension will be along the smallest axis of the structure. Semiconductor nanowires typically have dimensions of tens of nanometers in diameter and microns.

Nanowires are not observed spontaneously in nature and are produced in a laboratory. Nanowires may be formed from materials such as, for example, silicon, carbon, phosphorus, sulfur, nitrogen, aluminum, gold, silver, gallium, arsenic, titanium, germanium, indium, boron, cadmium, selenium, zinc, and the like, and mixtures, alloys, and chemical combinations thereof. Sulfur may be present as sulfide, and the like; nitrogen may be present as nitride and the like; selenium may be present as selenide and the like; phosphorus may be present as phosphide and the like; and so forth. Nanowires may be formed from semiconductor material such as, by way of illustration and not limitation, aluminum arsenide, aluminum gallium arsenide, boron nitride, cadmium sulfide, cadmium selenide, diamond, gallium arsenide, gallium nitride, germanium, indium phosphide, silicon, silicon carbide, silicon germanium, zinc sulfide, zinc selenide, and so forth, and combinations thereof.

Nanowires can be either suspended or deposited. A suspended nanowire is a wire in vacuum chamber held at the extremities. A deposited nanowire is a wire deposited on a surface of different nature. In some embodiments the nanowire can be a single strip of metallic atoms over a non-conducting surface. Nanowires can be grown substantially vertically on a substrate in a two-dimensional array format.

A suspended nanowire can be produced by chemical etching of a bigger wire, or bombarding a bigger wire with some highly energetic particles (atoms or molecules). Another way to produce a suspended nanowire is to indent a tip of an object in the surface of a metal near the melting point and retract it.

In some embodiments, nanowires are formed by placing nanoparticles on a surface that differs in composition from that of the nanoparticles. There are two common methods of placing nanoparticles on such a surface. In one approach a colloidal solution of a metal such as, for example, gold and the like is employed. The dilution of the solution determines the density of the nanoparticles on the surface and, thus, the size of the nanowire. The size of the nanowire relates to the spacing of the nanowires in the nanowire pair as discussed below. In another approach deposited thin films are annealed to a surface to form nanoparticles. The thickness of the film and the duration of the annealing process determine the density of the nanoparticle on the surface. Again, the size of the nanowire relates to the spacing of the nanowires in the nanowire pair as discussed below.

One technique for creating a nanowire is the Vapor-Liquid-Solid (VLS) synthesis method. This technique uses as source material either laser ablated particles or a feed gas. The source is then exposed to a catalyst. For some embodiments of nanowires, the catalysts are liquid metal (such as gold and the like) nanoclusters, which can either be purchased in colloidal form and deposited on a substrate or be self-assembled from a thin film by dewetting. This process can often produce crystalline nanowires in the case of semiconductor materials. The source enters the nanoclusters and begins to saturate it. Once supersaturation is reached, the source solidifies and grows outward from the nanocluster. The final product's length can be adjusted by simply turning off the source. Compound nanowires with super-lattices of alternating materials can be created by switching sources while still in the growth phase.

Another technique for creating a nanowire is to form a bridge between two substantially vertical surfaces. In one exemplary approach, highly oriented, metal-catalyzed silicon nanowires are grown laterally from vertical silicon planes and connection of the nanowires to other vertical silicon planes during growth. The silicon adjacent to the vertical planes can be further developed into electrodes. In this way, large numbers of nanowires in the form of nanobridges can be formed in parallel in a relatively small area or volume. The nanowires grow laterally from one vertical surface or sidewall to an opposing vertical surface.

An example of such a procedure is described by M. Saif Islam, et al., Nanotechnology 15 (2004) 1.5-1.8, who discuss ultrahigh density silicon nanobridges formed between two vertical silicon surfaces. A thermal oxide layer is first grown on oriented silicon wafers and patterned to serve as an etch mask for the subsequent silicon etch. The mask edges are carefully aligned along the intersection of vertical planes with the top surface so that the subsequently etched trench is bounded by two surfaces. The oxide is etched using reactive ion etching (RIE) with carbon trifluoride and argon gases and the silicon is etched in aqueous potassium hydroxide at elevated temperature such as, for example, 100° C. to 120° C., to create trenches of desired dimensions. After cleaning, nucleating metal catalyst, for example, titanium, gold or the like, of appropriate thickness, for example, one nanometer thick or the like, is deposited by electron beam evaporation onto the vertical surfaces of the etched trenches. In this exemplary approach, samples are held at an angle of about 45 degrees from the normal to deposit catalyst on only one sidewall of the trench so that the wires grow preferentially from one sidewall in this example. Such an orientation, however, is not critical and the growth of the nanowires may be accomplished in any particular orientation of the sample. Subsequently, the samples are transferred through air or non-reactive gas to a CVD reactor. In this particular example, the samples are supported by a silicon carbide-coated graphite plate of moderate thermal mass. The samples are annealed in hydrogen at a high temperature, for example, about 600° C. to about 650° C., to form gold-silicon alloy nanoparticles and to reduce the native oxide on the titanium and form TiSi₂. After a slight increase in temperature, for example, about 10° C. to about 20° C., a mixture of silicon tetrahydride and hydrochloric acid is introduced into the ambient hydrogen atmosphere for a period of time and under sufficient pressure (about 5 to about 15 Torr) to grow the nanowires.

The nanowires employed in the present invention comprise a p-n junction, which is generally known as a junction formed by combining N-type and P-type semiconductors together in very close contact. Although a p-n junction is preferred, other junctions are possible such as, for example, p-n-p and the like. The term junction refers to the region where the two types of semiconductors meet. For instance, a nanowire may comprise a metal such as, for example, titanium and n-doped silicon and p-doped silicon, all of which are present in a length appropriate for the nanowire. In some embodiments the nanowire may lie between a p-type mesa or an n-type mesa, which may be opposing substantially vertical walls of channel, or the like.

The well that comprises the plurality of nanowires may be part of a microfluidic device or component of a microfluidic system. The term “microfluidic device” as used herein refers to a device having fluidic conduit features, such as, e.g., channels, that are difficult or impossible to see with the naked eye, that is, having features on a scale of millimeters to tenths of micrometers. The term microfluidic device refers to a device having a network of chambers connected by channels, in which the channels have mesoscale dimensions, e.g., having at least one cross-sectional dimension in the range from about 0.1 μm to about 500 μm. In microfluidic devices, micro-volumes of fluid are manipulated along a fluid flow path. “Micro-volume” means a volume from about 10 femtoliters to 500 μl, usually from about 100 femtoliters to about 200 μl.

The number of nanowire pairs in the microfluidic device is based on a number of factors such as, for example, the complexity of the sample to be analyzed including the suspected number of analytes, specific application of the devices, and so forth. The number of nanowire pairs may be more than ten, more than one hundred, more than five hundred, more than one thousand, more than fifteen hundred, more than two thousand, more than twenty five hundred, more than 20,000, more than 25,000, more than 30,000, more than 35,000, more than 40,000, more than 50,000, more than 75,000, or more than 100,000. In many embodiments the number of nanowire pairs is in the range of about 10 to about 100, about 10 to about 1,000, about 100 to about 1,000, about 1,000 to about 10,000, about 100 to about 100,000, about 1000 to about 100,000, and so forth.

The spatial relationship or spacing between members of the p-n junction nanowire pair is dependent on such factors as the size of the nanowires, the ability of the detection nanowire to detect light from an excitation nanowire, the ability of the excitation nanowire to excite a luminescent label, and so forth. In some embodiments the spacing is in the range of about 1 to about 1,000 nm, about 1 to about 100 nm, about 10 to about 1,000 nm, about 10 to about 100 nm, and so forth. In this manner, each member of the nanowire pair is considered to be adjacent to the other member of the nanowire pair.

The spatial relationship or spacing between each pair of p-n junction nanowires is dependent on such factors as the size of the nanowires of the nanowire pairs, the space necessary to avoid interference between nanowires of one pair of nanowires with one or both nanowires of another nanowire pair, the specific application of the present device that determines power, size and volume requirements, and the like. In some embodiments the spacing is in the range of about 100 to about 100,000 nm, about 100 to about 10,000 nm, about 100 to about 1,000 nm, about 1,000 to about 100,000 nm, about 1,000 to about 10,000 nm, about 10,000 to about 100,000 nm, and so forth.

The microfluidic devices contain at least one fluid flow path through which fluid flows through the device, where a plurality of flow paths that may or may not be intersecting and may be positioned in any convenient configuration may be present in the device. Generally, the microfluidic devices have at least one chamber positioned at some point in the fluid flow path, where the term “chamber” means any type of structure in which micro-volumes of fluid may be contained, and includes micro-chambers, micro-channels, micro-conduits and the like. Depending on the nature of the chamber, the chamber may be the entire fluid flow path through the device, e.g., where the fluid flow path is a micro-channel, or may occupy only a portion of the fluid flow path of the device.

The term micro-chamber, as used herein, means any structure or compartment having a volume ranging from about 1 μl to about 500 μl, having cross-sectional areas ranging from about 0.05 cm² with a chamber depth of about 200 μm to about 5 cm² with a chamber depth of about 1 mm; or from about 10 μl to about 500 μl, having a cross-sectional area ranging from about 0.5 cm² with a chamber depth of about 200 μm to about 5 cm² with a chamber depth of about 1 mm; or from about 20 μl to about 200 μl, having a cross-sectional area ranging from about 1 cm² with a chamber depth of about 200 μm to about 4 cm² with a chamber depth of about 500 μm.

The chamber or channel structure may have any convenient configuration or cross-sectional shape, including square, circular, oval, trapezoidal, rectangular, octagonal, irregular, etc. Furthermore, the cross-section of the interior of a chamber or a channel may have several different cross-sectional shapes. For example, the cross-sectional shape of an area of the chamber or channel adjacent a pore or opening or orifice may be different than that of the remainder of the chamber.

Micro-channels or micro-conduits are chambers that are dimensioned or configured such that fluid is capable of flowing through the micro-channel by capillary flow, i.e., the micro-channel is of capillary dimensions. By capillary dimensions is meant a structure or container in which any cross-sectional dimension from one side to another, e.g., diameter, widest point between two walls of a channel, etc., does not exceed about 250 μm. Generally, for capillary flow, any cross-sectional dimension of the micro-channel will range from about 10 to about 250 μm, usually from about 50 to about 200 μm. The flow through the micro-channels may also be pressurized. Moving materials through microchannels may be accomplished by use of a fluid pressure difference and by use of various electro-kinetic processes including electrophoresis, electroosmotic flow, and electrokinetic pumping.

The micro-channel(s) of the device may have a linear configuration, a curved configuration, or any other configuration, e.g., spiral, angular, etc., or combinations thereof. In addition, as discussed above, there may be more than one micro-channel in the device, where the micro-channels may intersect at various points to form complicated flow paths or patterns through the device, e.g., Y-shaped intersections, T-shaped intersections, crosses; and/or be separated by one or more micro-chambers, etc.

In addition to a substrate that has features such as microfluidic channels, microfluidic compartments, and microfluidic flow control elements, the microfluidic component may include features such as capillary channels, separation channels, detection channels, valves and pumps. The microfluidic device may be a continuous or non-continuous flow device or a combination thereof. The devices also can include reservoirs, fluidly connected to the channels, which can be used to introduce material into the channels. Interfacing mechanisms, such as electropipettors, can be incorporated for transporting materials into wells or microfluidic channels.

In many embodiments, the micro-channel(s) of the microfluidic devices, as well as any other components, e.g., entry ports, etc., may be present in an essentially planar-shaped substrate, e.g., a card-shaped substrate, disk-shaped substrate, etc.

The materials from which the chambers and related components may be fabricated are dependent on the particular environment or use of the chamber, the nature of the liquid within the chamber, the advantages and limitations of particular fabrication techniques, and so forth. Materials for fabrication include polymers, plastics such as polyimides, polycarbonates, polyesters, polyamides, polyethers, polyolefins, and mixtures thereof, resins, polysaccharides, silica or silica-based or silicon dioxide based materials such as quartz, fused silica, glass (borosilicates) etc., ceramics and composites thereof, carbon, metals including metal alloys, metal oxides, inorganic glasses, and so forth and combinations thereof. Particular plastics finding use include, for example, polyethylene, polypropylene, such as high density polypropylene, polytetrafluoroethylene (PTFE), e.g., TEFLON®, polymethylmethacrylate, polycarbonate, polyethylene terephthalate, polystyrene or styrene copolymers, polyUrethanes, polyesters, polycarbonates, polyureas, polyamides, polyethyleneamines, polyarylene sulfides, polysiloxanes, polydimethylsiloxanes, polyimides, polyacetates, poly etheretherketone (PEEK), and the like. Metals include, for example, stainless steel, hastalloy, platinum, gold, silver, titanium, and so forth. Appropriate coatings for channels and chambers may be employed in order to achieve the desired excitation and detection properties of nanowires that may be disposed in the channels or chambers consistent with the present invention.

The microfluidic device or component may be fabricated by direct means such as photolithographic processes, wet or dry chemical etching, laser ablation, traditional machining and the like. The microfluidic component may also be fabricated by indirect means such as injection molding, hot embossing, casting, or other processes that utilize a mold or patterned tool to form the features of the microfluidic component.

The microfluidic devices may be fabricated as unitary devices or they may be constructed from several parts assembled into the device. Apertures may be made in the chamber housing by laser cutting, etching, piercing, drilling, punching, direct molding or casting from a master with pins, and so forth.

In one example, a microfluidic fluid system may include a microfluidic device having a fluid input and a fluid reservoir. The aforementioned devices may also include means for introducing liquids into the devices as well as means for moving materials in the liquids within the devices and means for providing electrical control of functions of the microfluidic device.

As mentioned above, one member of each of the nanowire pairs comprises a capture moiety, which is any moiety that is capable of binding to the analyte so that the analyte becomes associated with such member of the nanowire pair in a non-diffusive manner. In some embodiments, the capture moiety is a binding partner for one of the respective analytes, which are discussed in more detail below. The binding partner for the analyte depends on the nature of the analyte. Typical binding partners include, for example, antigens, antibodies, polynucleotide receptors, protein receptors, and the like.

Attaching the binding partner for the analyte to the nanowire may be accomplished in a number of different ways depending on the nature of the nanowire and the nature of the binding partner. The exposed surface of the nanowire, or of the well wall adjacent the nanowire, either has a functional group for attachment or must be treated or modified by chemical techniques to provide such a functional group or groups. Representative groups include, by way of illustration and not limitation, amino, especially primary amino, hydroxyl, thiol, sulfonic acid, phosphorous and phosphoric acid, particularly in the form of acid halides, especially chloride and bromide, and carboxyl, and the like.

A procedure for creating the attachment chemistry is sometimes referred to a “priming” the surface. To this end, the exposed surface is modified so as to prepare the surface for attachment of the binding partner. The binding partner may be attached directly to the exposed surface or it may be synthesized on the surface depending on the nature of the binding partner. In the former approach the binding partner comprises a functional group for attachment. In the latter approach the binding partner is formed in situ such as, for example, the formation of biopolymers by employing monomeric building blocks such as nucleotide triphosphates in the case of polynucleotides.

The exposed surface may be modified with groups or coupling agents to covalently link the binding partner or an initial monomeric unit. The reactive functional groups may be conveniently attached to the exposed surface through a hydrocarbyl radical such as an alkylene or phenylene divalent radical. Such hydrocarbyl groups may contain up to 10 carbon atoms, or up to 20 carbon atoms and the like.

In one embodiment, the surface of the nanowire is siliceous, i.e., the surface comprises silicon oxide groups, either present in the natural state, e.g., glass, silica, silicon with an oxide layer, etc., or introduced by techniques well known in the art. One technique for introducing siloxyl groups onto the surface involves reactive hydrophilic moieties on the surface. These moieties are typically epoxide groups, carboxyl groups, thiol groups, and/or substituted or unsubstituted amino groups as well as a functionality that may be used to introduce such a group such as, for example, an olefin that may be converted to a hydroxyl group by means well known in the art. One approach is disclosed in U.S. Pat. No. 5,474,796 (Brennan), the relevant portions of which are incorporated herein by reference. A siliceous surface may be used to form silyl linkages, i.e., linkages that involve silicon atoms. Usually, the silyl linkage involves a silicon-oxygen bond, a silicon-halogen bond, a silicon-nitrogen bond, or a silicon-carbon bond.

A procedure for the derivatization of a metal oxide surface uses an aminoalkyl silane derivative, e.g., trialkoxy 3-aminopropylsilane such as aminopropyltriethoxy silane (APS), 4-aminobutyltrimethoxysilane, 4-aminobutyltriethoxysilane, 2-aminoethyltriethoxysilane, and the like. APS reacts readily with the oxide and/or siloxyl groups on metal and silicon surfaces. APS provides primary amine groups that may be used to attach a binding partner to the nanowire. Such a derivatization procedure is described in EP 0 173 356 B1, the relevant portions of which are incorporated herein by reference. Other methods for treating the surface will be suggested to those skilled in the art in view of the teaching herein.

Embodiments of the present devices also comprise a mechanism for varying voltage applied to the p-n junction nanowires. In many embodiments the voltage to the nanowires is increased. The mechanism for varying applied voltage may comprise an electronics component comprising resistors, batteries, diodes, transistors, voltage regulators and the like. The mechanism may be, for example, a simple voltage regulator with a battery for portable applications, an AC to DC converter with a voltage regulator for non-portable applications, and so forth.

Whether the voltage to the nanowires is increased or decreased, and the amount of such increase or decrease, is dependent on the property of the nanowire that is employed in the determination. Increasing bias voltage of a p-n junction nanowire causes the p-n junction nanowire to emit light with a certain wavelength depending on the semiconductor materials. In these embodiments the member of the p-n junction nanowire pair that is employed to excite a luminescent label by, for example, emitting light, may be referred to as the excitation nanowire, which may be in the form, for example, of a photodiode nanowire or a laser nanowire or the like. The semiconductor material of the nanowire p-n junction is determined by the excitation wavelength of the luminescent labels, that is, the wavelength of light needed to excite the luminescent labels. The excitation wavelength of common luminescent labels is between about 400 to about 800 nm, although other labels having wavelengths of excitation less than or greater than the above range may also be utilized. In general, with the use of luminescent labels, the bias voltage of the p-n junction nanowires is increased such that light is emitted sufficient to excite the luminescent label or to respectively excite luminescent labels where more than one label is employed. Generally, luminescent labels are chosen such that the excitation wavelength and the emission wavelength are different.

The devices discussed above may include electronic and electrical processing support to enhance the capabilities of the system. The devices may include, for example, electronic and electrical processing support that perform operations such as voltage/current sourcing, signal sourcing, signal detection, signal processing, signal feedback, and data processing separately from the microfluidic system. The electronic processing and microfluidic functions may be separated or may be integrated. For example, a relatively large power supply is required in order to apply a high voltage to a microfluidic channel for electrophoresis, and it is best to locate the power supply separate from the microfluidic system. As another example, data analysis is best performed using a computer that is separate from the microfluidic system.

In some embodiments, the electronics component may provide for individually electrically addressing and/or reading each nanowire or groups of nanowires. Accordingly, a device in accordance with the present invention may comprise a plurality of electrical leads coupled to each of the nanowires for electrically individually addressing the nanowires. The electrical leads may be formed by any technique and material typically used for electrical connections in a thin-film circuit, being patterned and/or multi-layered structures of metals, doped semiconductors, conductive organic films, and the like.

On-system electrical processing may be employed in cases where information gathered from many sensors on a microfluidic system must be used to control processes on the microfluidic chip. For example, a temperature system input might be used to control heaters of a microfluidic system.

In addition to microfluidic features, the microfluidic device or component may include conductive traces that are formed within the substrate and/or on the surface of the substrate. The conductive traces provide electrical connection between the electronics component and various electrical features on or in the microfluidic component. These electrical features may include: (1) direct contacts to fluid; (2) elements which, either in contact with or not in contact with fluid, control the flow or the operation of fluid or its contents; (3) sensors in direct contact with fluid; (4) sensors that do not directly contact fluid; (5) electrical heating or cooling elements integrated in or on the microfluidic component; (6) elements that can affect surface change within the microfluidic component; (7) active microfluidic control elements such as valves, pumps, and mixers; and so forth. Conductive traces may also lead to contact pads on the microfluidic component that provide electrical connections to off-component systems such as signal processors, signal readout devices, power supplies, and/or data storage systems. Providing contact pads on the microfluidic component for connection to off-component systems may eliminate the need to provide such contact pads on the electronics component.

While the electronics component may be composed of discrete electrical elements on a common substrate, such as a conventional printed circuit board, the component may be a prefabricated integrated circuit that may perform any of a variety of functions. The prefabricated integrated circuit may include a combination of op-amps, transistors, diodes, multiplexers, switches, filters, logic, digital-to-analog converters, analog-to-digital converters, etc., that perform functions such as signal detection, signal processing, buffering, and/or control functions such as, e.g., flow control and the like. The electronics component can be, for example, an application specific integrated circuit. As an alternative to the integrated circuit chip, the electronics component may consist of discrete electrical devices mounted on a suitable substrate, such as a printed circuit board, which may be integral or non-integral with the microfluidic component. The electronics component may be fabricated separately from the microfluidic component utilizing conventional semiconductor processing techniques.

The electronics component may include signal processing circuitry. The signal processing circuitry may process signal from detection nanowires in accordance with the present methods. For example, the signal processing circuitry may amplify a signal, filter a signal, convert a signal from analog to digital, and make logical decisions based upon signal inputs. Because the possibilities for signal processing are numerous, it should be understood that any type of signal processing is anticipated for implementation in the electronics component consistent with the present methods involving detection of conductance or detection of luminescence. It should be understood that circuitry for detecting other phenomena may also be included within the electronics component.

The electronics component may also provide circuitry for control functions such as voltage control, current control, temperature control, clock signal generation, etc. Flow control circuitry may be incorporated in order to manipulate microfluidic flow control elements of the type previously identified (e.g., valves, pumps, and regulators). As with the detection and processing circuitry, the possibilities for control circuitry are numerous and therefore it should be understood that any type of control circuitry is anticipated for implementation in the electronics component.

The electronics component may also contain software or firmware that, through its operation, guides or controls the action of the circuitry. For example, the electronics component may contain programmable logic that allows a programmed algorithm to be executed so as to perform certain functions. These functions may include signal acquisition, signal filtration, signal feedback, control operations, signal interruption, and other forms of signal processing.

The electronics component may be fabricated in a separate operation utilizing either conventional semiconductor processing techniques or assembly of discrete electrical elements such as resistors, capacitors, operational amplifiers, and the like. The electronics component may include a combination of memory, signal detection, signal processing, and control circuitry. The control circuitry may provide voltage control, current control, temperature control, and/or clock signal generation. Where the electronics component is not integral with the microfluidic component, the electronics component can be bonded to the microfluidic component in various locations depending on the ease of manufacture and the like. In some embodiments, the electronic component may be maintained separate from the microfluidic device.

An integral or non-integral electronic component may assist in providing the necessary variation in voltage to the p-n junction nanowires in accordance with embodiments of the present methods as discussed herein.

Furthermore, an integral or non-integral electronic component may assist in processing signal from p-n junction detection nanowires. The detection nanowire is a nanowire that is employed to detect a signal produced as a result of the property of the excitation nanowire. As discussed herein, such signal may be luminescence, which results in a change in photocurrent of a photodetector p-n junction nanowire based on the binding of an analyte to a binding partner for the analyte. In some embodiments the signal may be a luminescent signal emitted by a luminescent label. In most embodiments, electrical connection is needed between an excitation nanowire and a voltage source and between a detection nanowire and an appropriate signal processor.

To assist in the automation of the present methods, the functions and methods may be carried out under computer control, that is, with the aid of a computer and computer program. The computer system is in communication with various components of the device and of the apparatus and the computer program product directs the components to carry out their respective functions.

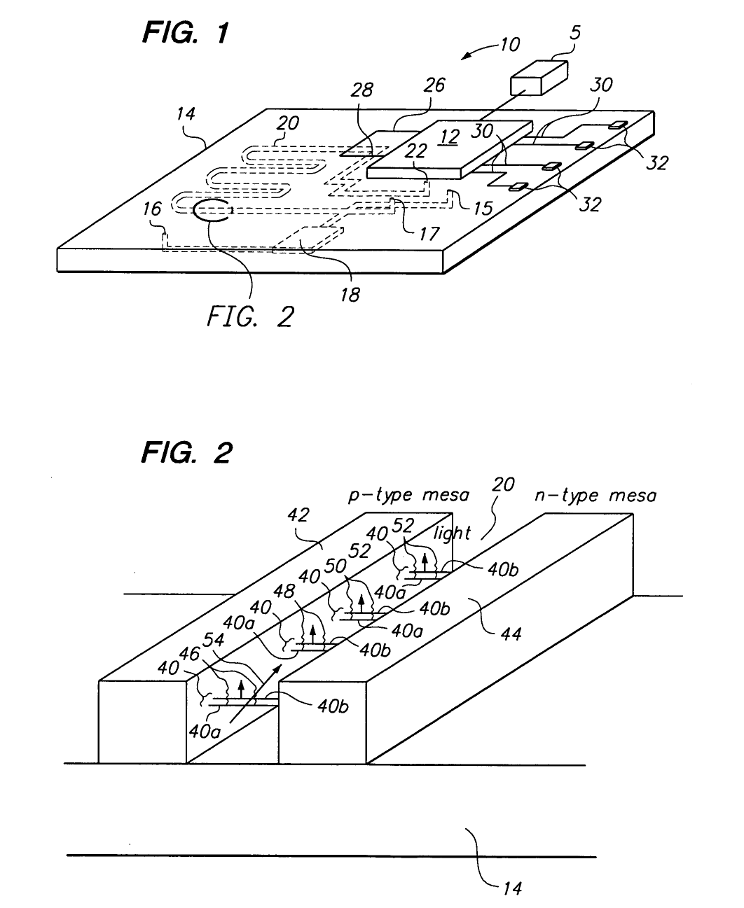

A specific embodiment of a device in accordance with embodiments of the present invention is shown in FIGS. 1 and 2. Referring specifically to FIG. 1, the microfluidic component 14 is a planar device that is part of apparatus 10 and includes chamber 18 having input/output ports 15 and 16 and further includes channel 20 having input/output ports 17 and 22. The chamber and channels are shown as dashed lines, since they are formed within the microfluidic component 14. The dashed lines are interrupted at the intersection of the channel from chamber 18 with channel 20 because the two channels intersect. Chamber 20 may be employed to carry out various sample preparation processes if required by a particular method. Such processes include, but are not limited to, mixing, labeling, filtering, extracting, precipitating, digesting, and the like. The microfluidic component also includes conductive traces 26, 28, and 30 that are formed within the substrate and/or on the surface of the substrate. For example, the conductive traces 26 and 28 may be used to assist in measure conductance as discussed above. The conductive traces 26 and 28 extend to the electronics component 12, which may be integral with or separate from microfluidic component 14. The microfluidic component also includes conductive traces 30 that connect the electronics component to contact pads 32. The contact pads may provide electrical connections to off-chip systems such as signal processors, signal readout devices, a power supply, and/or data storage systems as discussed above. Providing input/output contact pads on the microfluidic component is an alternative embodiment to providing such contact pads on the electronics component.

FIG. 2 shows a portion of channel 20, which comprises a plurality of nanowire pairs 40 each comprising nanowire 40a and 40b. Nanowire pairs 40 are disposed between walls 42 and 44 of channel 20. Wall 42 is a p-type mesa and wall 44 is an n-type mesa, thus, enabling each of nanowires 40 to function as p-n junction nanowires. Binding partners 46, 48, 50 and 52, each for a suspected analyte, are attached to respective nanowires 40a. Each of nanowires 40a and 40b is in electrical communication with electronics component 12.

The components of the present apparatus are adapted to perform a specified function usually by a combination of hardware and software. This includes the structure of the particular component and may also include a microprocessor, embedded real-time software and I/O interface electronics to control a sequence of operations and so forth.

The size of the overall device will depend on a number of factors such as the number of analytes, and thus the corresponding number of nanowires, area required by electronics, the particular manner in which the device is used, and the like.

### Methods

As mentioned above, embodiments of the present invention are directed to methods for analyzing a sample for the presence of one or more analytes. The sample is contacted with a well having contained therein a plurality of p-n junction nanowire pairs. One member of each of the nanowire pairs comprises a capture moiety such as, e.g., a binding partner for one of the respective analytes that may be present in the sample. The nature of the binding partners for the analytes is discussed above in detail.

In some embodiments the members of the nanowire pairs comprise p-n junction photoemission sources and photocurrent detectors. The contacting is carried out under conditions for binding of an analyte to a respective binding partner. The respective properties of photoemission and photocurrent detection are employed to determine the presence and/or amount of an analyte in the sample.

In some embodiments the members of the p-n junction nanowire pair comprise a functionalized p-n junction light emitting diode nanowire and a non-functionalized p-n junction photodetector nanowire. In these embodiments a nanowire is considered functionalized as a result of its ability to capture an analyte. The analytes bound to respective functionalized nanowires comprise a luminescent label and the property of the nanowire is light emission. The method comprises increasing the bias voltage of the functionalized p-n junction nanowires such that light is emitted sufficient to excite each of the luminescent labels and determining the amount of luminescence emitted from the luminescent labels by the adjacent non-functionalized p-n junction photodetector nanowire. The amount of luminescence is related to the presence and/or amount of one or more of the analytes in the sample.

In some embodiments the p-n junction nanowire pairs comprise a non-functionalized p-n junction light emitting diode and a functionalized p-n junction photodetector. The method comprises increasing the bias voltage of the non-functionalized p-n junction nanowires such that light is emitted sufficient to excite each of the luminescent labels. The method comprises measuring the photocurrent of each of the functionalized p-n junction photodetector nanowires and relating the amount of photocurrent at each of the functionalized nanowires to the presence and/or amount of an analyte in the sample. The analytes bound to a respective functionalized nanowire each comprise a luminescent label. The amount of luminescence is related to the presence and/or amount of one or more of the analytes in the sample.

The analytes are the compounds or compositions to be detected. The analyte is usually comprised of a member of a specific binding pair (sbp) and its binding partner, which is the other member of the specific binding pair. The analyte or the binding partner may be a ligand, which is monovalent (monoepitopic) or polyvalent (polyepitopic), usually antigenic or haptenic, and is a single compound or plurality of compounds that share at least one common epitopic or determinant site. The analyte can be a part of a cell such as a bacterium or a cell bearing a blood group antigen such as A, B, D, etc., or an HLA antigen or the analyte may be a microorganism, e.g., bacterium, fungus, protozoan, or virus. In certain circumstances the analyte may also be a reference compound, a control compound, a calibrator, and the like.

The monoepitopic ligand analytes will generally be from about 100 to about 2,000 molecular weight, more usually, from about 125 to about 1,000 molecular weight. The monoepitopic analytes include drugs, e.g., drugs of abuse and therapeutic drugs, metabolites, pesticides, pollutants, nucleosides, and the like. Included among drugs of interest are the alkaloids, steroids, lactams, aminoalkylbenzenes, benzheterocyclics, purines, drugs derived from marijuana, hormones, vitamins, prostaglandins, tricyclic antidepressants, anti-neoplastics, aminoglycosides, antibiotics, nucleosides and nucleotides, miscellaneous individual drugs which include methadone, meprobamate, serotonin, meperidine, lidocaine, procainamide, acetylprocainamide, propranolol, griseofulvin, valproic acid, butyrophenones, antihistamines, chloramphenicol, anticholinergic drugs, such as atropine, their metabolites and derivatives, and so forth.

Metabolites related to diseased states include spermine, galactose, phenylpyruvic acid, and porphyrin Type 1 and so forth.

Among pesticides of interest are polyhalogenated biphenyls, phosphate esters, thiophosphates, carbamates, polyhalogenated sulfenamides, their metabolites and derivatives.

The polyvalent ligand analytes will normally be poly(amino acids), i.e., polypeptides and proteins, polysaccharides, mucopolysaccharides, nucleic acids, and combinations thereof. Such combinations include components of bacteria, viruses, chromosomes, genes, mitochondria, nuclei, cell membranes and the like.

A polynucleotide or nucleic acid is a compound or composition that is a polymeric nucleotide or nucleic acid polymer, which may include modified nucleotides.

For the most part, the polyepitopic ligand analytes to which the subject invention can be applied have a molecular weight of at least about 5,000, more usually at least about 10,000. In the poly(amino acid) category, the poly(amino acids) of interest will generally be from about 5,000 to 5,000,000 molecular weight, more usually from about 20,000 to 1,000,000 molecular weight; among the hormones of interest, the molecular weights will usually range from about 5,000 to 60,000 molecular weight.

A wide variety of proteins may be considered as to the family of proteins having similar structural features, proteins having particular biological functions, proteins related to specific microorganisms, particularly disease causing microorganisms, etc. Such proteins include, for example, immunoglobulins, cytokines, enzymes, hormones, cancer antigens, nutritional markers, tissue specific antigens, etc. Such proteins include, by way of illustration and not limitation, protamines, histones, albumins, globulins, scleroproteins, phosphoproteins, mucoproteins, chromoproteins, lipoproteins, nucleoproteins, glycoproteins, T-cell receptors, proteoglycans, HLA, unclassified proteins, e.g., somatotropin, prolactin, insulin, pepsin, proteins found in human plasma, blood clotting factors, protein hormones such as, e.g., follicle-stimulating hormone, luteinizing hormone, luteotropin, prolactin, chorionic gonadotropin, tissue hormones, cytokines, cancer antigens such as, e.g., PSA, CEA, a-fetoprotein, acid phosphatase, CA19.9 and CA125, tissue specific antigens, such as, e.g., alkaline phosphatase, myoglobin, CPK-MB and calcitonin, and peptide hormones. Other polymeric materials of interest are mucopolysaccharides and polysaccharides.

For receptor analytes, the molecular weights will generally range from 10,000 to 2×10⁸, more usually from 10,000 to 10⁶. For immunoglobulins, IgA, IgG, IgE and IgM, the molecular weights will generally vary from about 160,000 to about 10⁶. Enzymes will normally range from about 10,000 to 1,000,000 in molecular weight. Natural receptors vary widely, generally being at least about 25,000 molecular weight and may be 10⁶ or higher molecular weight, including such materials as avidin, DNA, RNA, thyroxine binding globulin, thyroxine binding prealbumin, transcortin, etc.

The term analyte further includes polynucleotide analytes such as those polynucleotides defined below. These include m-RNA, r-RNA, t-RNA, DNA, DNA-RNA duplexes, etc. The term analyte also includes receptors that are polynucleotide binding agents, such as, for example, restriction enzymes, activators, repressors, nucleases, polymerases, histones, repair enzymes, chemotherapeutic agents, and the like.

Also included within the term “analyte” are polysaccharides or carbohydrates, lipids, fatty acids and the like.

The analyte may be a biomarker, which is a biochemical feature or facet that can be used to measure the progress of a disease or illness or the effects of treatment of a disease or illness. The biomarker may be, for example, a virus, a bacteria, a cancer antigen, a heart disease indicator, a stroke indicator, an Alzheimer's disease indicator, and the like.

The analytes may be molecules found directly in a sample such as biological tissue, including body fluids, from a host. The sample can be examined directly or may be pretreated to render the analytes more readily detectable. Furthermore, the analytes of interest may be determined by detecting agents probative of the analytes of interest such as a specific binding pair member complementary to the analyte of interest, whose presence will be detected only when a particular analyte of interest is present in a sample. Thus, the agent probative of the analyte becomes the analyte that is detected in an assay.

The sample may be a “body fluid sample” or a “non-body fluid sample.” The phrase “body fluid sample” refers to any fluid obtained from the body of a mammal (e.g., human, monkey, mouse, rat, rabbit, dog, cat, sheep, cow, pig, and the like), bird, reptile, amphibian or fish that is suspected of containing a particular target analyte or analytes to be detected. The biological tissue includes excised tissue from an organ or other body part of a host and body fluids. Exemplary body fluid samples for detection herein can be selected from one or more of whole-blood, plasma, serum, interstitial fluid, sweat, saliva, urine, semen, blister fluid, inflammatory exudates, stool, sputum, cerebral spinal fluid, tears, mucus, and the like. Also explicitly contemplated herein as a “body fluid sample” are body gas and body vapor. The phrase “non-body fluid sample” refers to any fluid not obtained from the body of a mammal, bird, reptile, amphibian or fish, which is suspected of containing a particular target analyte or analytes to be detected. Exemplary non-body fluid samples include cell culture media, artificial collection fluid, dialysate, and the like. An artificial collection fluid (or extraction fluid) can be prepared by bathing a particular surface area of an animal or an inanimate object with a fluid to collect into the fluid an endogenous or exogenous analyte for detection.

For determining a mixture of analytes such as, for example, proteins, one may use intact cells, intact viruses, viral infected cells, lysates, plasmids, mitochondria or other organelles, fractionated samples, or other aggregation of analytes, separated analytes, and treated analytes, by themselves or in conjunction with other compounds. Any source of a mixture of analytes can be used, where there is an interest in identifying a plurality of analytes. Analytes may be released and/or isolated using precipitation, extraction, lysing, chromatographic separation, and so forth and combinations thereof. The analytes may be present as individual analytes or combined in various aggregations, such as organelles, cells, viruses, etc.

The concentration of analytes to be detected will generally vary from about 10⁻⁵ to 10⁻¹⁷ M, more usually from about 10⁻⁶ to 10⁻¹⁴ M.

The medium suspected of containing the analytes, which may or may not comprise a luminescent label, is contacted with the well comprising nanowire pairs wherein one member of the nanowire pairs has a capture moiety such as, e.g., a binding partner for a respective analyte. In some embodiments the medium is an aqueous medium and in other embodiments the medium is a non-aqueous medium. The nature of the medium depends on the nature of the analytes and the like.

An aqueous medium may be solely water or may include from 0.01 to 80 or more volume percent of a cosolvent such as an organic solvent, which may be polar or non-polar, usually polar for purposes of solubility. Examples of polar organic solvents include oxygenated organic solvents of from 1 to about 30 carbon atoms, or 1 to about 20 carbon atoms, or 1 to about 10 carbon atoms including alcohols, ethers, ketones, aldehydes, amides, nitriles, and so forth. Particular examples include alcohols such as, e.g., ethoxyethanol, ethanol, ethylene glycol and benzyl alcohol; amides such as dimethyl formamide, formamide, acetamide and tetramethyl urea and the like; sulfoxides such as dimethyl sulfoxide and sulfolane; nitriles such as, e.g., acetonitrile, and so forth, ethers such as carbitol, ethyl carbitol, dimethoxyethane, and the like. Non-polar solvents include, for example, hydrocarbons containing 1 to about 30 carbon atoms, or 1 to about 20 carbon atoms, or 1 to about 10 carbon atoms, and so forth; halogenated hydrocarbons such as, e.g., methylene chloride, trichloromethane carbon tetrachloride, and so forth.

When an aqueous medium is employed, it is generally an aqueous buffered medium that is buffered at a moderate pH, generally that which provides optimum sensitivity and specificity for a particular analyses. The pH for the medium will usually be in the range of about 4 to 13, more usually in the range of about 5 to 10, and preferably in the range of about 6.5 to 9.5. Various buffers may be used to achieve the desired pH and maintain the pH during the determination. Illustrative buffers include borate, phosphate, carbonate, tris, barbital and the like. The particular buffer employed is not critical, but in an individual analyses one or another buffer may be preferred.

As mentioned above, the medium suspected of containing the analytes, which may or may not comprise a luminescent label, is contacted with the well comprising pairs of nanowires wherein one member of the pair has a respective binding partner for one of the analytes. In some embodiments the well is part of a microfluidic device and takes the form of one or more chambers or channels within the microfluidic device. In many embodiments, the medium is introduced into the microfluidic device by means of capillary action. However, as mentioned above, other forms of introduction may be employed such as, for example, application of positive or negative pressure, and the like

The contacting is carried out under conditions for binding of an analyte to a respective binding partner. Moderate temperatures are normally employed and, in many embodiments, the temperature is usually a constant temperature. The temperatures for binding will normally range from about 5° to about 99° C., about 15° to about 70° C., about 20 to about 45° C. Temperatures during measurements will generally range from about 10° to about 70° C., about 20° to about 45° C., about 20° to about 25° C. It will be appreciated, however, that higher or lower temperatures may be employed depending on the nature of the analytes, binding partners, medium, and the like.

Subsequent to the binding reactions, a property of the nanowires is employed to determine the presence and/or amount of an analyte in the sample. The property of the nanowire may be, for example, photoemission, photodetection, and the like. In some embodiments, one of the members of the p-n junction nanowire pair has the property of being a photoemitter, or a source of photoemission, and the other member of the nanowire pair has the property of being a photodetector, for example, a photocurrent detector. One of the members of the nanowire pair, either the photoemitter or the photodetector, may comprise a capture moiety.

The term “photoemission” means emission of light from a p-n junction nanowire by raising the bias voltage of the nanowire.

The term “photoemitter” means a p-n junction nanowire that emits light by raising the bias voltage of the nanowire.

The term “photodetection” means the ability of a p-n junction nanowire to detect light (e.g., photons) and produce corresponding photocurrent.

The term “photodetector” means a p-n junction nanowire that has the ability to detect light (e.g., photons) and produce corresponding photocurrent.

The term “photocurrent” means current that flows through a p-n junction nanowire as a result exposure to light. In some embodiments, light results from excitation of a luminescent label.

In some embodiments the p-n junction nanowire photodetector is a photodiode, which means that the nanowire photodetector has the property or ability to be responsive to light. Light exposure (e.g., photons) produces hole and electron pairs inside a photodiode and create corresponding photocurrent in the photodiode. The magnitude of the photocurrent depends on the number of photons captured by the photodiode.

In most embodiments each of the analytes bound to a respective nanowire comprises a luminescent label. In these embodiments, the analytes are treated to introduce a luminescent label prior to or after contacting a medium suspected of containing the analytes with the well comprising the nanowires having binding partners for the analytes attached thereto.

Luminescence is a phenomenon in which energy is specifically channeled to a molecule to produce an excited state. Return to a lower energy state is accompanied by release of a photon. Luminescence includes fluorescence, phosphorescence, chemiluminescence and bioluminescence. In some embodiments, the luminescent labels may be spectrally distinguishable from one another to achieve differential detection of analytes.

In some embodiments, the luminescent label may be a fluorescent label or a phosphorescent label. The fluorescent labels may be excited to fluoresce by exposure to certain wavelengths of light. A fluorescent label is a molecule which, following absorption of light of wavelengths of about 250 to about 1100 nm, preferably about 300 to about 950 nm, emits light by fluorescence or phosphorescence, preferably by fluorescence. In some embodiments the fluorescent compounds absorb light between about 300 and about 800 nanometers and emit between about 400 and about 1000 nanometers. Examples of suitable fluorescent labels, by way of illustration and not limitation, include polycyclic aromatic hydrocarbons such as anthracenes, e.g., bisphenylethynylanthracene; coumarins; naphthacenes; phthalocyanines; squaraines, e.g., bis-(4-dimethlyaminophenyl)squaraine; porphyrins; polyacetylenes, oxazine dyes; rare earth chelates, e.g., Eu, Tb and Sm, etc.; xanthenes such as rhodamine and fluorescein; coumarins such as umbelliferone; aromatic amines such as dansyl; squarate dyes; benzofurans; cyanines; merocyanines; chromenes; and the like. Particular examples of compounds include, for example, 5-TMRIA (tetramethylrhodamine-5-iodoacetamide), Quantum Red.TM., Texas Red.TM., Cy3, N-((2-iodoacetoxy)ethyl)-N-methyl)amino-7-nitrobenzoxadiazole (IANBD), 6-acryloyl-2-dimethylaminonaphthalene (acrylodan), pyrene, Lucifer Yellow, Cy5, Dapoxyl.RTM. (2-bromoacetamidoethyl) sulfonamide, (N-(4,4-difluoro-1,3,5,7-tetramethyl-4-bora-3a,4a-diaza-s-indacene-2-yl), N-(4,4-difluoro-5,7-diphenyl-4-bora-3a,4a-diaza-s-indacene-3-propionyl)-N′-iodoacetylethylenediamine, 5-((((2-iodoacetyl)amino)ethyl)amino)naphthalene-1-sulfonic acid, and carboxy-X-rhodamine, 5/6-iodoacetamide, 2,5-diphenyloxazole (PPO), anthracene, 2-(4′-tert-butylphenyl)-5-(4″-biphenyl)-1,3,4-oxadiazole; 1-phenyl-3-mesityl-2-pyrazoline; rare earth metal cryptate allopycocyanin, allophycocyanin B, phycocyanin C or phycocyanin R, thiomine, phycocyanin R, phycoerythrocyanin, phycoerythrin C, phycoerythrin B, phycoerythrin R; Eu trisbipyridine diamine (EuTBP) and Tb tribipyridine diamine (TbTBP).

As mentioned above, in some embodiments the analytes are treated to attach a respective luminescent label prior to or after being exposed to the nanowires comprising binding partners for the respective analytes that might be present in a medium to be analyzed. Attachment of a luminescent label to an analyte may be accomplished directly or indirectly, covalently or non-covalently. Covalent attachment may be by a bond (direct attachment) or a linking group (indirect attachment). In either case, covalent attachment normally involves one or more functional groups on the luminescent label and/or the analyte. In embodiments where a linking group is involved, the linking group varies depending upon the nature of the molecules, i.e., the luminescent label or the analyte. Functional groups that are normally present or are introduced on the molecules to be attached are employed for linking these materials.

Alternative functionalities of oxo include active halogen, diazo, mercapto, olefin, particularly activated olefin, amino, phosphoro and the like. The linking groups may vary from a bond to a chain of from 1 to 100 atoms, usually from about 1 to 70 atoms, preferably 1 to 50 atoms more preferably 1 to 20 atoms, each independently selected from the group normally consisting of carbon, oxygen, sulfur, nitrogen, halogen and phosphorous. The number of heteroatoms in the linking groups will normally range from about 0 to 20, usually from about 1 to 15, more preferably 2 to 6. The atoms in the chain may be substituted with atoms other than hydrogen in a manner similar to that described above for the substitutent having from 1 to 50 atoms. As a general rule, the length of a particular linking group can be selected arbitrarily to provide for convenience of synthesis and the incorporation of the desired luminescent label. The linking groups may be aliphatic or aromatic, although with diazo groups, aromatic groups will usually be involved.

When heteroatoms are present, oxygen will normally be present as oxo or oxy, bonded to carbon, sulfur, nitrogen or phosphorous, nitrogen will normally be present as nitro, nitroso or amino, normally bonded to carbon, oxygen, sulfur or phosphorous; sulfur would be analogous to oxygen; while phosphorous will be bonded to carbon, sulfur, oxygen or nitrogen, usually as phosphonate and phosphate mono- or diester.

Common functionalities in forming a covalent bond between the linking group and the molecule to be conjugated are alkylamine, amidine, thioamide, ether, urea, thiourea, guanidine, azo, thioether and carboxylate, sulfonate, and phosphate esters, amides and thioesters. For the most part, carbonyl functionalities will find use, both oxocarbonyl, e.g., aldehyde, and non-oxocarbonyl (including nitrogen and sulfur analogs) e.g., carboxy, amidine, amidate, thiocarboxy and thionocarboxy.

In some embodiments, the linking group has a non-oxocarbonyl group including nitrogen and sulfur analogs, a phosphate group, an amino group, alkylating agent such as halo or tosylalkyl, oxy (hydroxyl or the sulfinur analog, mercapto) oxocarbonyl (e.g., aldehyde or ketone), or active olefin such as a vinyl sulfone or α-, β-unsaturated ester. These functionalities will be linked to amine groups, carboxyl groups, active olefins, alkylating agents, e.g., bromoacetyl. Where an amine and carboxylic acid or its nitrogen derivative or phosphoric acid are linked, amides, amidines and phosphoramides will be formed. Where mercaptan and activated olefin are linked, thioethers will be formed. Where a mercaptan and an alkylating agent are linked, thioethers will be formed. Where aldehyde and an amine are linked under reducing conditions, an alkylamine will be formed. Where a carboxylic acid or phosphate acid and an alcohol are linked, esters will be formed.

Non-covalent attachment of a luminescent label may involve a luminescent label being bound to a binding partner for the analyte such as, for example, an antibody or other receptor for the analyte, and the like. The binding partner chosen for attachment of a luminescent label is normally different from the binding partner that is attached to the nanowire. The two binding partners at least should be different enough to bind to different sites on the analyte. The binding partner with the luminescent label attached may be combined with the analyte prior to or after contact of the medium suspected of containing the analyte with the nanowires. As a further alternative, the analyte, for example, may be bound by a first antibody specific to the analyte, while the luminescent label is a labeled second antibody specific to the first antibody. Other approaches will be suggested to one skilled in the art in light of the present disclosure.

Following exposure of the medium to the nanowires and incubation under conditions for binding of the analytes to respective binding partners attached to the nanowires, a wash fluid may be introduced into and flowed through the microfluidic device to remove unbound materials. However, in some instances a wash fluid is not required because each nanowire provides for its own localized detection site.

Following the above, the method involves increasing the bias voltage of the p-n junction nanowires such that light is emitted sufficient to excite each of the luminescent labels. In that regard an electronic component as discussed above may be employed to assist in increasing the bias voltage at respective nanowires and, thus, individually addressing each nanowire or groups of nanowires. The extent of the increase of bias voltage as well as the duration is, in general, that which is sufficient to emit light of sufficient magnitude and duration to excite each of the luminescent labels. The duration of the increase in bias voltage may be about 0.01 to about 30 seconds, or about 0.10 to about 20 seconds, or the like.

Detection of luminescence based on the binding of an analyte to a respective binding partner is, therefore, achieved with assistance of the photodetector nanowire member of the p-n junction nanowire pair in conjunction with the electronic component.

In accordance with some embodiments, the bias voltage of the member of the p-n junction nanowire pair that does not comprise a capture moiety is increased such that light is emitted sufficient to excite the luminescent label on the analyte captured by the member of the nanowire pair that comprises a capture moiety. The excited label emits luminescence that results in a signal, which is detected by the member of the nanowire pair that comprises the capture moiety. In these embodiments the member of the p-n junction nanowire pair that does not comprise a capture moiety may be referred to as a photoemitter and the member of the nanowire pair that comprises the capture moiety may be referred to as a photodetector. In some embodiments the photodetector nanowire detects photocurrent, which is produced by the luminescence emitted by the excited luminescent label.

In accordance with some embodiments, the bias voltage of the member of the p-n junction nanowire pair that comprises a capture moiety is increased such that light is emitted sufficient to excite the luminescent label on the analyte captured by the member of the nanowire pair that comprises a capture moiety. The excited label emits luminescence that results in a signal, which is detected by the member of the nanowire pair that does not comprise the capture moiety. In these embodiments the member of the p-n junction nanowire pair that comprises a capture moiety may be referred to a photoemitter and the member of the nanowire pair that does not comprise the capture moiety may be referred to as a photodetector.

Signals are conveyed to a central computer with the assistance of the electronic component where the signals are analyzed and related to the presence of an analyte at a particular member of a nanowire pair. Luminescence at a particular nanowire is correlated with the presence of an analyte bound to a respective binding partner at the particular member of the nanowire pair. In many embodiments the identity of each binding partner at each nanowire is known so that the analytes may be differentially detected and/or quantitated. Quantitation may be realized by measuring the amount of luminescence or photocurrent at a particular nanowire and relating the amount of thereof to the amount of analyte in the sample.

A particular embodiment of a method in accordance with the present invention will be discussed, by way of illustration and not limitation, with reference to FIGS. 1 and 2. A sample suspected of containing one or more analytes is treated to introduce a luminescent label, in this instance a fluorescent label, on each of the analytes. A medium comprising the sample treated as above is contacted with port 16 of microfluidic system 10 and allowed to flow into microfluidic device 14. The medium travels along a flow path 54 defined by channel 20 and analytes, if present, from the medium bind to respective binding partners, a portion of which includes binding partners 46, 48, 50, 52 attached to nanowires 40a, respectively. For purposes of this example, assume that an analyte is present that binds to binding partners 46, 48, 50, 52 so that a fluorescently labeled analyte is bound at nanowires 40 that comprise binding partners 46, 48, 50, 52, respectively. The flow rate, temperature and the like of the medium are sufficient to permit the binding reactions to occur.

Following the binding of the analytes to respective binding partners and the passage of a wash fluid if necessary, the bias voltage at each of nanowires 40b is increased by means of electronic component 12 so that nanowires 40b emit light of a wavelength sufficient to respectively excite the fluorescent label or labels of the analytes attached to each of the respective binding partners including binding partners 46, 48, 50, 52. In this example, nanowires 40b have the property of being a photoemitter. Each of the excited fluorescent labels emits light of a wavelength different from the excitation wavelength. The emitted light or signal is detected by a respective member 40a of p-n junction nanowire pair 40, which in this example has the property of being a photodetector and photocurrent that is produced by the light emitted from the fluorescent labels is measured by members 40a. Each p-n junction nanowire pair is in electronic communication with electronic component 12, which assists in relaying the signal from each nanowire pair member 40a to computer 56, which correlates the signal to the presence and/or amount of the respective analytes in the sample.

Another particular embodiment of a method in accordance with the present invention will be discussed, by way of illustration and not limitation, again with reference to FIGS. 1 and 2. A sample suspected of containing one or more analytes is treated to introduce a luminescent label, in this instance a fluorescent label, on each of the analytes. A medium comprising the sample treated as above is contacted with port 16 of microfluidic system 10 and allowed to flow into microfluidic device 14. The medium travels along a flow path 54 defined by channel 20 and analytes, if present, from the medium bind to respective binding partners, a portion of which includes binding partners 46, 48, 50, 52 attached to nanowires 40a, respectively. For purposes of this example, assume that an analyte is present that binds to binding partners 46, 48, 50, 52 so that a fluorescently labeled analyte is bound at nanowires 40 that comprise binding partners 46, 48, 50, 52, respectively. The flow rate, temperature and the like of the medium are sufficient to permit the binding reactions to occur.

Following the binding of the analytes to respective binding partners and the passage of a wash fluid if necessary, the bias voltage at each of nanowires 40a is increased by means of electronic component 12 so that nanowires 40a emit light of a wavelength sufficient to respectively excite the fluorescent label or labels of the analytes attached to each of the respective binding partners including binding partners 46, 48, 50, 52. In this example, nanowires 40a have the property of being a photoemitter. Each of the excited fluorescent labels emits light of a wavelength different from the excitation wavelength. The emitted light or signal is detected by a respective member 40b of p-n junction nanowire pair 40, which in this example has the property of being a photodetector and photocurrent that is produced by the light emitted from the fluorescent labels is measured by members 40b. Each p-n junction nanowire pair is in electronic communication with electronic component 12, which assists in relaying the signal from each nanowire pair member 40b to computer 56, which correlates the signal to the presence and/or amount of the respective analytes in the sample.

### Apparatus

As mentioned above, one embodiment of the present invention is an apparatus comprising a microfluidic system including a microfluidic device as described above, a computer system, which comprises a computer, for controlling the mechanism for varying the bias voltage of the p-n junction nanowires, and a computer program on a computer readable medium for controlling the computer.

The computer may be, for example, an IBM® compatible personal computer (PC) and the like. The computer is driven by software specific to the methods described herein. Software that may be used to carry out the methods may be, for example, Microsoft Excel or Microsoft Access and the like, suitably extended via user-written functions and templates, and linked when necessary to stand-alone programs that perform other functions. The computer system is in communication with various components of the device and of the apparatus and the computer program product directs the components to carry out their respective functions.

The computer system may be programmed from a computer readable storage medium that carries code for the system to execute the steps required of it, thus, having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically or optically and including, for example, machine readable bar code, solid state electronic storage devices such as random access memory (RAM), or read only memory (ROM), or any other physical device or medium that might be employed to store a computer program. It will also be understood that computer systems of the present invention can include the foregoing programmable systems and/or hardware or hardware/software combinations that can execute the same or equivalent steps. Accordingly, stored programming embodying steps for carrying-out the subject methods may be transferred to a computer such as a personal computer (PC), (i.e., accessible by a researcher or the like), by physical transfer of a CD, floppy disk, or like medium, or may be transferred using a computer network, server, or other interface connection, e.g., the Internet.

The computer program product, therefore, comprises a computer readable storage medium having a computer program stored thereon which, when loaded into a computer, performs the aforementioned method and/or controls the functions of the aforementioned apparatus.

The computer program is designed to carry out a method for analyzing a sample for the presence of one or more analytes. The computer program provides for carrying out steps in a method wherein a sample is contacted with a well, such as by flowing therethrough, having contained therein a plurality of p-n junction nanowire pairs, wherein one member of each of the nanowire pairs comprises a capture moiety for one of the respective analytes and wherein the contacting is carried out under conditions for binding of an analyte to a respective binding partner and wherein a property of one of the members of the nanowire pairs is employed to determine the presence and/or amount of an analyte in the sample.

In some embodiments the property of the nanowire is photocurrent and the computer program product provides for measuring the photocurrent detected by one member of the p-n junction nanowire pairs and relating the amount of photocurrent to the presence and/or amount of an analyte in the sample.

In some embodiments each of the analytes bound to a respective member of a nanowire pair comprises a luminescent label. The computer program product provides for increasing the bias voltage of a member of the p-n junction nanowire pair such that light is emitted sufficient to excite each of the luminescent labels and determining the amount of luminescence emitted from the luminescent labels by a photodetector nanowire wherein the amount thereof is related to the presence and/or amount of one or more of the analytes in the sample.

An embodiment of an apparatus in accordance with the present invention is depicted in FIG. 1 by way of illustration and not limitation. Apparatus 10 comprises microfluidic component 14, electronics component 12 and computer 56.

All publications and patent applications cited in this specification are herein incorporated by reference as if each individual publication or patent application were specifically and individually indicated to be incorporated by reference, except insofar as they may conflict with those of the present application (in which case the present application prevails). Methods recited herein may be carried out in any order of the recited events, which is logically possible, as well as the recited order of events.

The aforementioned description includes theories and mechanisms by which the invention is thought to work. It should be noted, however, that such proposed theories and mechanisms are not required and the scope of the present invention should not be limited by any particular theory and/or mechanism.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for analyzing a sample for the presence of one or more analytes, said method comprising:
(a) contacting the sample with a well comprising a plurality of p-n junction nanowire pairs, each of the nanowire pairs comprising an excitation nanowire and a detection nanowire wherein one member of the nanowire pair comprises a capture moiety and wherein the contacting is carried out under conditions for binding of an analyte to a respective capture moiety, and
(b) using the excitation nanowire to excite a luminescent label bound to the capture moiety and the detection nanowire to detect a signal resulting from excitation of the luminescent label, wherein the signal is related to the presence and/or amount of an analyte in the sample.

2. A method according to claim 1 wherein the member of the nanowire pair comprising the capture moiety is the excitation nanowire and the other member of the nanowire pair is the detection nanowire and the method comprises increasing the bias voltage of the excitation nanowire sufficient to excite the luminescent label and detecting luminescence emitted by the luminescent label by means of the detection wire.

3. A method according to claim 1 wherein the member of the nanowire pair comprising the capture moiety is the detection nanowire and the other member of the nanowire pair is the excitation nanowire and the method comprises increasing the bias voltage of the excitation nanowire to excite the luminescent label and detecting photocurrent by means of the detection nanowire.

4. A method for analyzing a sample for the presence of one or more analytes, said method comprising:
(a) contacting the sample with a well comprising a plurality of p-n junction nanowire pairs, wherein one member of the nanowire pair comprises a capture moiety and wherein the contacting is carried out under conditions for binding of an analyte to a respective capture moiety, and
(b) using a light emitting property of one member of the nanowire pair and a detection property of the other member of the nanowire pair to determine the presence and/or amount of an analyte in the sample.

5. A method according to claim 4 wherein the detection property is detection of photocurrent.

6. A method according to claim 4 wherein the capture moiety is a binding partner for one of the respective analytes and wherein each of the analytes bound to a respective capture moiety comprises a luminescent label and the method comprises increasing the bias voltage of the p-n junction nanowire comprising the capture moiety such that light is emitted sufficient to excite each of the luminescent labels and detecting the amount of luminescence emitted from the luminescent labels by means of measuring photocurrent using the other member of the p-n junction nanowire pair, the amount of luminescence being related to the presence and/or amount of one or more of the analytes in the sample.

7. A method according to claim 6 wherein the luminescent labels are fluorescent compounds.

8. A method according to claim 4 wherein the capture moiety is a binding partner for one of the respective analytes and wherein each of the analytes bound to a respective capture moiety comprises a luminescent label and the method comprises increasing the bias voltage of the other member of the p-n junction nanowire pairs such that light is emitted sufficient to excite each of the luminescent labels on the member of the p-n junction nanowire pairs comprising the capture moiety and detecting photocurrent by means of each member of the p-n junction nanowire pair comprising a capture moiety, the amount of light being related to the presence and/or amount of one or more of the analytes in the sample.

9. A method according to claim 8 wherein the luminescent labels are fluorescent compounds.

10. A method according to claim 4 wherein the well is a channel in the substrate.

11. A method according to claim 10 wherein the channel is part of a microfluidic system.

12. A method according to claim 10 wherein the contacting is carried out by passing the sample through the channel.

13. A method according to claim 4 wherein the analytes are selected from the group consisting of small organic compounds, proteins, peptides, higher molecular weight carbohydrates, polynucleotides, fatty acids and lipids.

14. A method for analyzing a sample for the presence of one or more analytes, said method comprising:
(a) contacting the sample with a channel of a microfluidic system wherein the channel comprises a plurality of p-n junction nanowire pairs and wherein one member of the nanowire pair comprises a capture moiety that is a binding partner for an analyte and wherein the contacting is carried out under conditions for binding of an analyte to a respective binding partner, and wherein each of the analytes comprises a luminescent label,
(b) increasing the bias voltage of one of the members of the p-n junction nanowire pairs such that light is emitted sufficient to excite each of the luminescent labels and
(c) determining the luminescence emitted from each of the luminescent labels by means of a photocurrent detection property of one of the members of the nanowire pairs, the luminescence being related to the presence and/or amount of one or more of the analytes in the sample.

15. A method according to claim 14 wherein the luminescent labels are fluorescent labels.

16. A method according to claim 14 wherein the analytes are selected from the group consisting of small organic compounds, proteins, peptides, higher molecular weight carbohydrates, polynucleotides, fatty acids and lipids.

17. A method according to claim 14 wherein the analytes are biomarkers.

18. A method according to claim 17 wherein the biomarkers are selected from the group consisting of viruses, bacteria, cancer antigens, disease indicators and infectious agents.

19. A method according to claim 14 wherein the method comprises increasing the bias voltage of the p-n junction nanowire comprising the capture moiety such that light is emitted sufficient to excite each of the luminescent labels and detecting the amount of luminescence emitted from each of the luminescent labels by means of measuring photocurrent using the other member of the p-n junction nanowire pair, the amount of luminescence being related to the presence and/or amount of one or more of the analytes in the sample.

20. A method according to claim 19 wherein the luminescent labels are fluorescent compounds.

21. A method according to claim 14 wherein the method comprises increasing the bias voltage of the other member of the p-n junction nanowire pairs such that light is emitted sufficient to excite each of the luminescent labels on the member of the p-n junction nanowire pairs comprising the capture moiety and detecting photocurrent by means of each member of the p-n junction nanowire pair comprising a capture moiety, the amount of photocurrent being related to the presence and/or amount of one or more of the analytes in the sample.

22. A method according to claim 21 wherein the luminescent labels are fluorescent compounds.

23. A device for analyzing a sample for the presence of one or more analytes, said device comprising:
(a) a well comprising a plurality of p-n junction nanowire pairs wherein one member of each of the nanowire pairs comprises a binding partner for one of the respective analytes and wherein one member of each of the nanowire pairs is a photocurrent detector and one member is light emitter, and
(b) a mechanism for varying the bias voltage of the light emitter member of the p-n junction nanowire pairs such that light is emitted.

24. A device according to claim 23 wherein the well is a channel in the substrate.

25. A device according to claim 24 wherein the channel is part of a microfluidic system.

26. A device according to claim 23 wherein the mechanism is a mechanism for varying the bias voltage of the member of the p-n junction nanowire pairs comprising the binding partner for one of the respective analytes.

27. A device according to claim 23 wherein the mechanism is a mechanism for varying the bias voltage of the member of the p-n junction nanowire pairs other than the member comprising the binding partner for one of the respective analytes.

28. An apparatus comprising:
(a) a device according to claim 23,
(b) a computer system for controlling mechanism for varying the bias voltage of the p-n junction nanowires, and
(c) a computer program on a computer readable medium.
